(19) United States
(12) Patent Application Publication
(10) Pub. No.: US 2023/0277170 A1
(43) Pub. Date: Sep. 7, 2023

(54) CONDUCTING SUTURES AND METHODS FOR FORMING AND USING SAME

(71) Applicants: Dion Khodagholy, Cresskill, NJ (US); Onni Johannes Rauhala, New York, NY (US)

(72) Inventors: Dion Khodagholy, Cresskill, NJ (US); Onni Johannes Rauhala, New York, NY (US)

(21) Appl. No.: 18/118,044

(22) Filed: Mar. 6, 2023

Related U.S. Application Data
(60) Provisional application No. 63/316,986, filed on Mar. 5, 2022.

(51) Int. Cl. A61B 17/06; A61L 17/14

(52) U.S. Cl. CPC A61B 17/06166; A61L 17/145; A61B 2017/00526

(57) ABSTRACT

Sutures including a surgical suture material and a conducting coating that has been applied to the surgical suture material are provided, wherein an electrical conductivity of the suture greater is than 500 S/cm per 1964 μm² of cross-sectional area of the suture. In some instances, an electro-chemical impedance of the suture is less than 10 kOhms at 1 kHz per 1964 μm² of cross-sectional area of the suture. In some instances, a diameter of the suture is 50-100 μm. In some instances, the conducting coating includes PEDOT:PSS. In some instances, the conducting coating further includes metal particles or metal flakes. In some instances, the suture further comprises: an insulating layer. In some instances, the insulating layer comprises at least one of Parylene-C, polyurethane, and a silicone elastomer. In some instances, the insulating layer was applied to the surgical suture material after the conducting coating was applied to the surgical suture material.

### CROSS-REFERENCE TO RELATED APPLICATION

This application claims the benefit of U.S. Patent Application No. 63/316,986, filed Mar. 5, 2022, which is hereby incorporated by reference herein in its entirety.

### BACKGROUND

Although advances in wireless signal acquisition systems have reduced the invasiveness of many implantable medical and research devices, wire-based systems continue to be necessary for applications directed at treating or analyzing certain areas of the body that are incompatible with wireless device use or conventional surgical approaches. Traditional metal wires provide the high conductivity that is needed for in vivo electrophysiological signal transmission. However, due to their rigidity, metal-based wires are susceptible to mechanical damage and discomfort to the subject.

Accordingly, conducting sutures are desirable.

### SUMMARY

In accordance with some embodiments, conducting sutures and methods for forming and using same are provided.

In accordance with some embodiments, conducting and biocompatible sutures are provided that can be concomitantly used for wound suturing, electrophysiological signal acquisition (e.g., for ECG, EMG, and LPF data collection), electrophysiological signal transmission, power delivery, muscle stimulation (e.g., to evoke movements), and/or ionic delivery. In some embodiments, these conducting and biocompatible sutures can be used in applications that require the suture to conduct electricity and/or ions between inside the body and outside the body. In some embodiments, these conducting and biocompatible sutures can be used on humans and other animals.

In some embodiments, sutures are provided, the sutures comprising: a surgical suture material; and a conducting coating that has been applied to the surgical suture material, wherein an electrical conductivity of the suture is greater than 500 S/cm per 1964 μm² of cross-sectional area of the suture. In some of these embodiments, an electro-chemical impedance of the suture is less than 10 kOhms at 1 kHz per 1964 μm² of cross-sectional area of the suture. In some of these embodiments, a diameter of the suture is 50-100 μm. In some of these embodiments, the conducting coating includes PEDOT:PSS. In some of these embodiments, the conducting coating further includes metal particles or metal flakes. In some of these embodiments, the suture further comprises: an insulating layer. In some of these embodiments, the insulating layer comprises at least one of Parylene-C, polyurethane, and a silicone elastomer. In some of these embodiments, the insulating layer was applied to the surgical suture material after the conducting coating was applied to the surgical suture material. In some of these embodiments, the suture is capable of being used for wound suturing. In some of these embodiments, the suture is capable of being used for electrophysiological signal acquisition, electrophysiological signal transmission, or electrical stimulation. In some of these embodiments, the suture is capable of being used for power delivery. In some of these embodiments, the suture is capable of being used for ion delivery.

In some of these embodiments, methods for forming a suture are provided, the methods comprising: coating a surgical suture material with a conducting coating to form a conducting suture, wherein an electrical conductivity of the conducting suture is greater than 500 S/cm per 1964 μm² of cross-sectional area of the suture. In some of these embodiments, an electro-chemical impedance of the conducting suture is less than 10 kOhms at 1 kHz per 1964 μm² of cross-sectional area of the conducting suture. In some of these embodiments, a diameter of the conducting suture is 50-100 μm. In some of these embodiments, the conducting coating includes PEDOT:PSS. In some of these embodiments, the conducting coating further includes metal particles or metal flakes. In some of these embodiments, the methods further comprise forming an insulating layer on the conducting suture. In some of these embodiments, the insulating layer comprises at least one of Parylene-C, polyurethane, and a silicone elastomer. In some of these embodiments, the insulating layer was applied to the surgical suture material after the conducting coating was applied to the surgical suture material. In some of these embodiments, the methods further comprise using the conducting suture for wound suturing. In some of these embodiments, the methods further comprise using the conducting suture for electrophysiological signal acquisition, electrophysiological signal transmission, or electrical stimulation. In some of these embodiments, the methods further comprise using the conducting suture for power delivery. In some of these embodiments, the methods further comprise using the conducting suture for ion delivery.

### BRIEF DESCRIPTION OF THE DRAWINGS

FIG. 1 is an example of a process for cleaning a suture in accordance with some embodiments.

FIG. 2 is an example of an image of a cleaned suture in accordance with some embodiments.

FIG. 3 is an example of a process for coating a suture in accordance with some embodiments.

FIG. 4 is an example of an image of a coated suture in accordance with some embodiments.

FIG. 5 is an example of an image of a coated suture with metal particles or flakes in accordance with some embodiments.

FIG. 6 is an example of process for applying a jacket (insulator) for a coated suture in accordance with some embodiments.

FIG. 7 is an example of image of an image of a coated suture with a jacket in accordance with some embodiments.

FIG. 8 is an example of a graph showing conductivity of a suture based on the number of coats of conducting solution applied to the suture in accordance with some embodiments.

FIG. 9 is an example of a graph of resistance and conductivity of a suture based on the length of the suture in accordance with some embodiments.

FIG. 10 is an example of a graph of impedance to an area surrounding a suture when provided with different jackets and no jacket in accordance with some embodiments.

FIG. 11 is an example of using a conducting suture for ion delivery in accordance with some embodiments.

### DETAILED DESCRIPTION

In accordance with some embodiments, conducting and biocompatible sutures are provided that can be concomitantly used for wound suturing, electrophysiological signal acquisition (e.g., for ECG, EMG, and LPF data collection), electrophysiological signal transmission, power delivery, muscle stimulation (e.g., to evoke movements), and/or ionic delivery. In some embodiments, these conducting and biocompatible sutures can be used in applications that require the suture to conduct electricity and/or ions between inside the body and outside the body. In some embodiments, these conducting and biocompatible sutures can be used on humans and other animals.

In accordance with some embodiments, a solution of the conducting polymer PEDOT:PSS together with solvents and a cross-linking agent can be used to coat surgical sutures and make them electrically and ionically conductive.

In some embodiments, the electrical conduction of the sutures is further improved by coating them metal particles or flakes (e.g., gold flakes). In some embodiments, this metal particle coating increases the suture conductivity.

In some embodiments, an insulating layer (jacket) for the sutures can be implemented using polyurethane, silicone elastomer (e.g., polydimethylsiloxane (PDMS)), Parylene-C, and/or using any other suitable substance.

In some embodiments, the conducting sutures described herein have a variety of beneficial properties including that they are biocompatible, soft, have a low electro-chemical impedance (e.g., less than 10 kOhms at 1 kHz per 1964 μm² of cross-sectional area of the conducting suture, have a high electrical conductivity (e.g., greater than 500 S/cm per 1964 μm² of cross-sectional area of the conducting suture), and have a small diameter (e.g., 50-100 μm). In some embodiments, these beneficial properties enable an end of a conducting suture as described herein to be used as a probe or an electrode. In some of these embodiments, these beneficial properties, such as biocompatibility, enable using the conducting suture for wound suturing. In some of these embodiments, these beneficial properties, such as conductivity, enable using the conducting suture for electrophysiological signal acquisition, electrophysiological signal transmission, or electrical stimulation. In some of these embodiments, these beneficial properties, such as conductivity, enable using the conducting suture for power delivery. In some of these embodiments, these beneficial properties, such as conductivity, enable using the conducting suture for ion delivery.

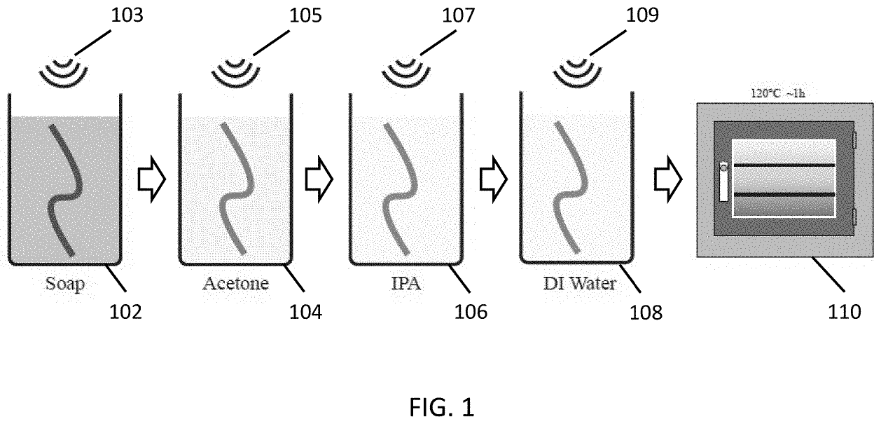

FIGS. 1-7 show an example of a process for fabricating electrically conducting sutures in accordance with some embodiments.

Turning to FIG. 1, a raw suture (meaning a suture before it is processed as described herein) can be prepared for coating as follows.

Any suitable suture can be used as the raw suture in some embodiments. For example, in some embodiments medical grade sutures such as silk, polysaccharide, polyester, and/or nylon medical grade sutures can be used as the raw suture in some embodiments.

First, at 102, the raw suture can be soaked in a soap-water solution for a first period of time to clean the suture, in some embodiments. Any suitable soap water solution can be used in some embodiments. For example, in some embodiments, surfactants (e.g., such as 4-Dodecylbenzenesulfonic acid) can be used. In some embodiments, while soaking in the soap-water, the suture can also be subjected to ultrasonic vibrations 103. Any suitable ultrasonic vibrations can be used in some embodiments. The raw suture can be soaked in soap-water and/or subjected to ultrasonic vibrations for any suitable first period of time in some embodiments. For example, in some embodiments, the first period of time can be 15 minutes.

Second, at 104, the suture can next be soaked in acetone for a second period of time, in some embodiments. Any suitable acetone can be used in some embodiments. For example, in some embodiments, semi-conducting grade acetone can be used. In some embodiments, while soaking in the acetone, the suture can also be subjected to ultrasonic vibrations 105. Any suitable ultrasonic vibrations can be used in some embodiments. The suture can be soaked in acetone and/or subjected to ultrasonic vibrations for any suitable second period of time in some embodiments. For example, in some embodiments, the second period of time can be 15 minutes.

Third, at 106, the suture can then be soaked in isopropyl alcohol (IPA) for a third period of time, in some embodiments. Any suitable IPA can be used in some embodiments. For example, in some embodiments, semi-conducting grade IPA can be used. In some embodiments, while soaking in the IPA, the suture can also be subjected to ultrasonic vibrations 107. Any suitable ultrasonic vibrations can be used in some embodiments. The suture can be soaked in IPA and/or subjected to ultrasonic vibrations for any suitable third period of time in some embodiments. For example, in some embodiments, the third period of time can be 15 minutes.

Fourth, at 108, the suture can next be soaked in deionized water for a fourth period of time in some embodiments. Any suitable deionized water can be used in some embodiments. In some embodiments, while soaking in the deionized water, the suture can also be subjected to ultrasonic vibrations 109. Any suitable ultrasonic vibrations can be used in some embodiments. The suture can be soaked in deionized water for any suitable fourth period of time in some embodiments. For example, in some embodiments, the fourth period of time can be 15 minutes.

Fifth, at 110, the suture can then be heated for a fifth period of time in some embodiments. The suture can be heated in a chamber at any suitable temperature in some embodiments. For example, in some embodiments, the suture can be heated in a chamber that has an air temperature of approximately 120 degrees Celsius. The suture can be heated in a chamber for any suitable period of time in some embodiments. For example, in some embodiments, the suture can be heated in a chamber for approximately one hour.

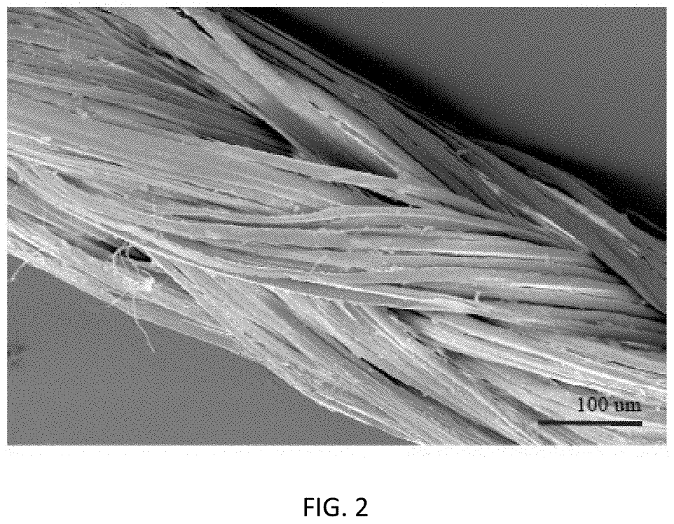

FIG. 2 shows an example of an image of a cleaned suture in accordance with some embodiments.

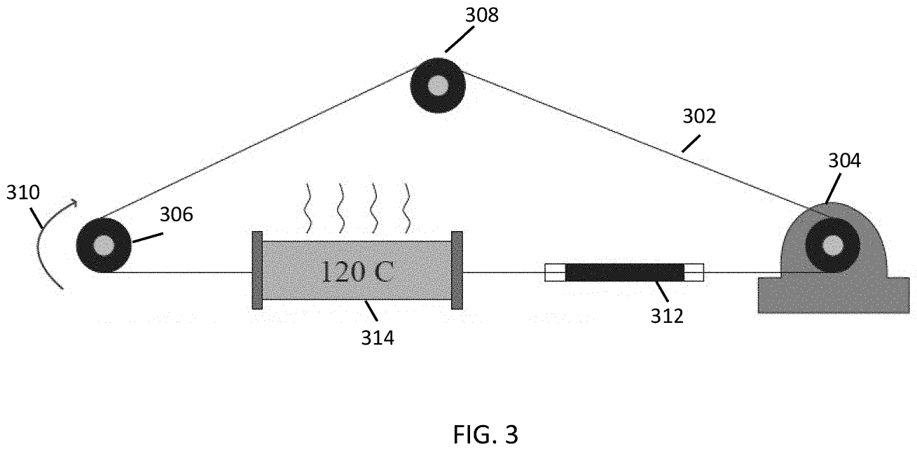

Turning to FIG. 3, a cleaned suture can be coated as follows.

First, the cleaned suture can be formed into a loop 302 around two or more rotation points 304, 306, and 308. Any suitable length of loop 302 can be used in some embodiments. For example, in some embodiments, the loop can have a length of 70 cm. Any suitable number of two or more rotation points 304, 306, and 308 can be used in some embodiments. For example, in some embodiments, two, three, four, or any other suitable number of two or more rotation points can be used. Any suitable mechanism(s) can be used for the two or more rotation points in some embodiments. For example, in some embodiments, the two or more rotation points can be pulleys, rollers, etc. Any suitable one or more of the two or more rotation points can cause the cleaned loop to move around the rotation points in any suitable manner in some embodiments. For example, in some embodiments, one 304 of the two or more rotation points can be a motorized (or motor driven) pulley. The loop can be under any suitable tension in some embodiments. The loop can move (as shown by 310) at any suitable speed in some embodiments. For example, in some embodiments, the loop can be moved at 1 rpm.

Second, the loop can be exposed to a conductive solution in a container 312 in some embodiments. Any suitable conductive solution can be used in some embodiments. For example, in some embodiments, the conductive solution can be a combination PEDOT:PSS, ethylene glycol, dodecylbenzenesulfonic acid (DBSA), (3-glycidyloxypropyl) trimethoxysilane (GOPS). Any suitable ratio(s) of the components in the conductive solution can be used in some embodiments. For example, in some embodiments, the components in the conductive solution be as follows (or any suitable scaling of same):
PEDOT:PSS—20 ml+/−5 ml;
ethylene glycol—5 ml+/−1 ml;
DBSA—1% volume by volume of the PEDOT:PSS and the ethylene glycol; and
GOPS—0.5% volume by volume of the PEDOT:PSS and the ethylene glycol.
   The loop can be exposed to the conductive solution in any suitable manner in some embodiments. For example, in some embodiments, the loop can be exposed to the conductive solution by soaking the loop in the conductive solution in container 312, by spraying the conductive solution onto the loop while in container 312, and/or by pressing the conductive solution into the loop via rollers. The conductive solution can be exposed to the conductive solution for any suitable period of time in some embodiments. For example, in some embodiments, the loop can be exposed to the conductive solution for 10-15 seconds per coat or revolution. This can be achieved in some embodiments by loop 302 moving (as shown by 310) through a distance of conductive solution exposure (e.g., in a bath or a spray in container 312) at a rate that equates to each exposed point of the loop having been exposed to the conductive solution for the desired time.

Third the loop can be heated in a container 314 in some embodiments. Any suitable manner of heating the loop can be used in some embodiments. For example, in some embodiments, the loop can be heated by passing it through hot air in container 314. Any suitable temperature air, such as air at 120 degrees Celsius, can be used in some embodiments. As another example, in some embodiments, the loop can be heated by exposing it to any suitable source of energy in container 314 that will heat the loop, such as infrared light and heat radiators. The loop can be heated to any suitable temperature in some embodiments. For example, in some embodiments, the loop can be heated to 120 degrees Celsius. Any suitable duration of heating the loop can be used in some embodiments. For example, in some embodiments, the loop can be heated for 10-15 seconds per coat or revolution.

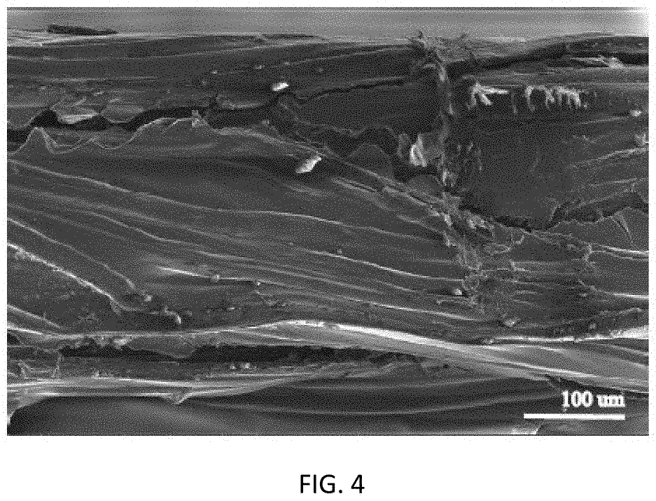

FIG. 4 shows an example of an image of a coated suture in accordance with some embodiments.

In some embodiments, a conductive solution containing metal particles or flakes can additionally or alternatively be used to coat the suture. Any suitable metal particles or flakes can be used in some embodiments. For example, in some embodiments, gold particles or flakes can be used. Any suitable size particles or flakes can be used in some embodiments. For example, in some embodiments, metal flakes can be 10-100 μm wide by less than 100 nm thick.

The metal particles or flakes can be formed in any suitable manner in some embodiments. For example, in some embodiments, the metal particles or flakes can be formed by soaking a silicon wafer having the metal thereon in acetone for a period of time, in some embodiments. Any suitable acetone can be used in some embodiments. For example, in some embodiments, semi-conducting grade acetone can be used. In some embodiments, while soaking in the acetone, the silicon wafer can also be subjected to ultrasonic vibrations. Any suitable ultrasonic vibrations can be used in some embodiments. The silicon wafer can be soaked in acetone and/or subjected to ultrasonic vibrations for any suitable period of time in some embodiments. For example, in some embodiments, the period of time can be 15-20 minutes. Once the metal particles or flakes are in suspension in the acetone, the silicon wafer can be removed and the metal particles or flakes extracted from the acetone.

In some embodiments, the metal particles or flakes can be added to a combination of PEDOT:PSS and Sorbitol. Any suitable ratio(s) of the components in the conductive solution can be used in some embodiments. For example, in some embodiments, the components in the conductive solution be as follows:
PEDOT:PSS;
Sorbitol—40% weight by volume of the PEDOT:PSS; and
Metal Particles or Flakes—10-50% volume by volume of the PEDOT:PSS.

The conductive solution with metal particles or flakes can be applied to the suture in addition to, or alternatively to, the conductive solution described above in connection with FIG. 3. When the conductive solution with metal particles or flakes is applied to the suture in addition to the conductive solution described above in connection with FIG. 3, the conductive solution with metal particles or flakes can be applied to the suture after any suitable number of coats of the conductive solution described above in connection with FIG. 3 by replacing the conductive solution described above in connection with FIG. 3 being applied in container 312 with the conductive solution with metal particles or flakes for any suitable number of coats. When the conductive solution with metal particles or flakes is applied to the suture alternatively to the conductive solution described above in connection with FIG. 3, the conductive solution with metal particles or flakes can replace the conductive solution described above in connection with FIG. 3 in the process described above in connection with FIG. 3 for any suitable number of coats.

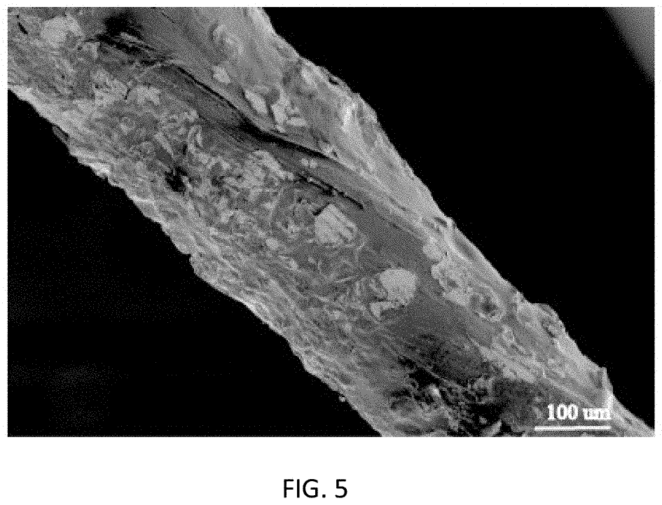

FIG. 5 shows an example of an image of a suture coating with a conductive solution including metal particles or flakes in accordance with some embodiments.

In some embodiments, a jacket can be applied to the coated suture described herein. Any suitable jacket can be used in some embodiments. For example, in some embodiments, a jacket made of polyurethane, a silicon elastomer (e.g., polydimethylsiloxane (PDMS)), or Parylene C can be used in some embodiments. A jacket can be applied in any suitable manner in some embodiments. For example, in some embodiments, a jacket of polyurethane or a silicon elastomer can be applied with an applicator such as a brush or cotton swab. As another example, in some embodiments, a jacket of Parylene C can be applied by stirring a coated suture in the Parylene C or by applying the Parylene C to the coated suture using chemical vapor deposition.

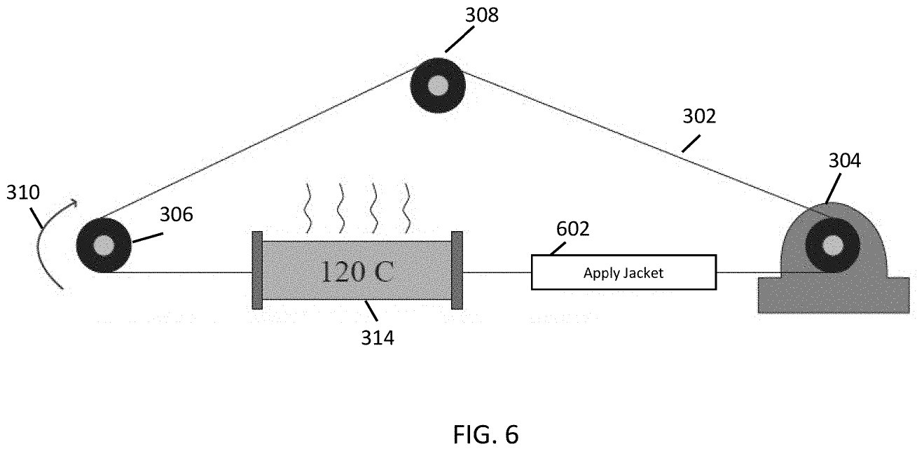

FIG. 6 illustrates an example of applying a jacket to a coated suture in accordance with embodiment some embodiments. After a suture has been coated as described above in connection with FIG. 3, a jacket can be applied as described above at 602 to the coated suture. In some embodiments, after applying the jacket, the jacketed suture can be heated. Any suitable manner of heating the jacketed suture can be used in some embodiments. For example, in some embodiments, the jacketed suture can be heated by passing it through hot air in container 314. As another example, in some embodiments, the loop can be heated by exposing it to any suitable source of energy in container 314 that will heat the jacketed suture, such as infrared light or heat radiator. The jacketed suture can be heated to any suitable temperature in some embodiments. For example, in some embodiments, the jacketed suture can be heated to 80 degrees Celsius. Any suitable duration of heating the jacketed suture can be used in some embodiments. For example, in some embodiments, the jacketed suture can be heated in container 314 for 10-15 seconds per coat or revolution.

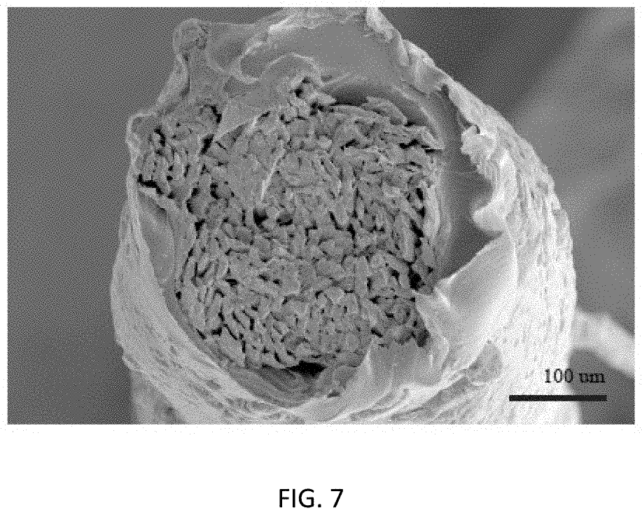

FIG. 7 shows an example of an image of a jacketed suture in accordance with some embodiments.

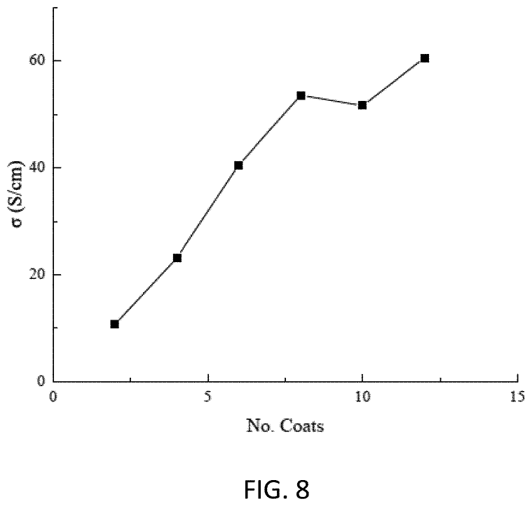

FIG. 8 illustrates an example of conductivity values a (in Siemens/cm) that can be realized for different coated sutures based on the number of coats of a conductive solution applied to the sutures in accordance with some embodiments. As shown, the conductivity of the coated sutures generally goes up based on the number of coats applied to the suture.

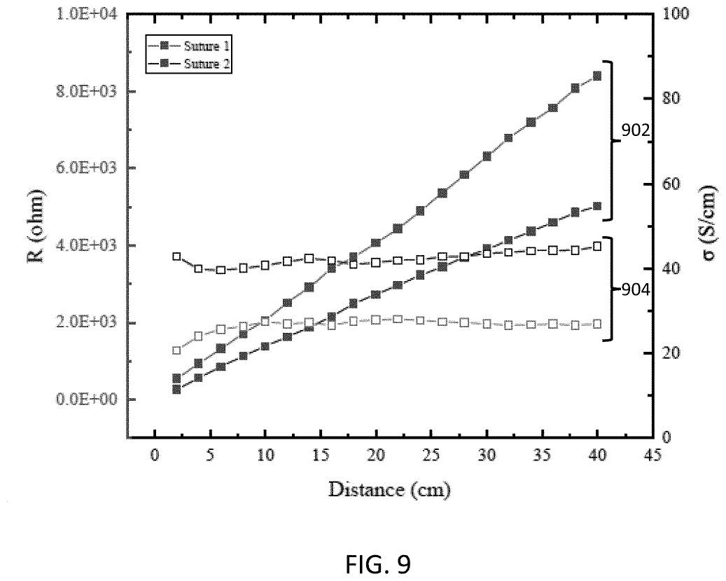

FIG. 9 illustrates an example of resistance values R (in ohms) 902 and conductivity values σ (in Siemens/cm) 904 that can be realized for different lengths of coated sutures in accordance with some embodiments. As shown, the resistance values for coated sutures generally increase linearly with increasing length similar to how an ordinary wire's resistance generally increases linearly. As also shown, the conductivity remains generally constant with increasing length (since conductivity is measure in Siemens per cm of length) similar to how an ordinary wire's conductivity generally remains constant.

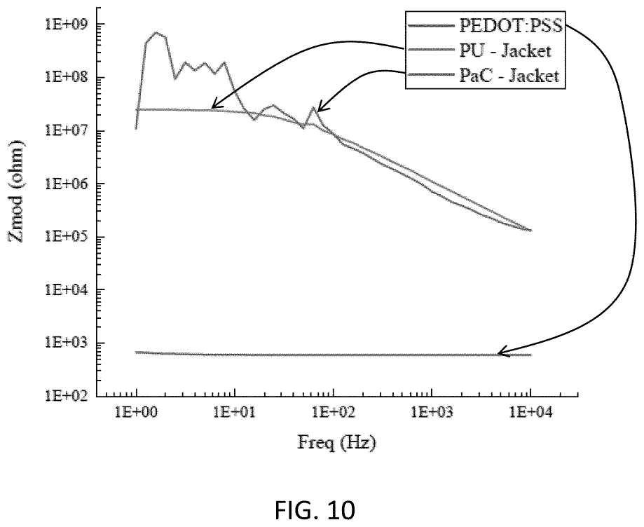

FIG. 10 illustrates an example of the impedance Zmod (in ohms) of unjacketed conducting sutures (marked in the figure as “PEDOT:PSS”), conducting sutures jacketed with polyurethane (marked in the figure as “PU”), and conducting sutures jacketed with Parylene C (marked in the figure as “PaC”) to areas adjacent to the conducting sutures but not intended to be in electrical contact with the conducting sutures, in accordance with embodiment some embodiments. As shown, the jacketed sutures perform much better than the unjacketed suture in electrically insulating the sutures from the surrounding areas.

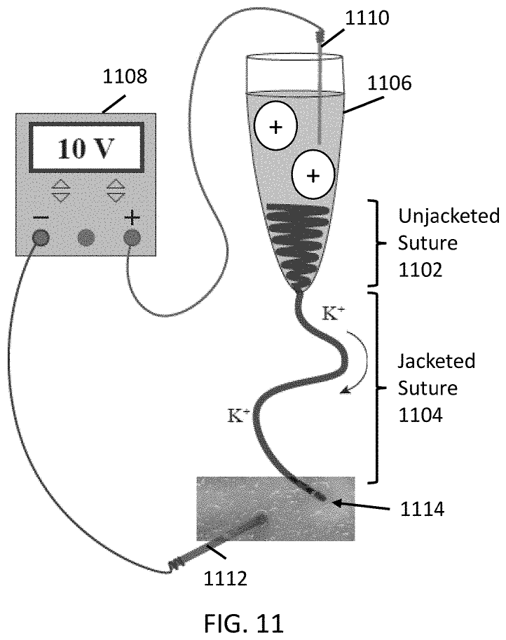

Turning to FIG. 11, an illustration of using conducting sutures for ionic transfer in accordance with some embodiments is shown. This ionic transfer can be used for any suitable purpose such as to deliver medication to a desired area in some embodiments.

As illustrated in FIG. 11, a combined suture that is a combination of an unjacketed suture 1102 and a jacketed suture 1104 can be used in some embodiments. This combined suture can be prepared in any suitable manner such as by connecting an unjacketed suture to an unjacketed suture, or by jacketing only a portion of a coated suture, in some embodiments. The unjacketed suture portion and the jacketed suture portion can be prepared as described above in some embodiments.

The unjacketed suture can be placed in a reservoir 1106 of an ionic solution, as represented by (+) in the figure. Any suitable ionic solution can be used, and the ionic solution can have any suitable ions in it. For example, the ionic solution can be a therapeutic, and the ions can be charged molecules of a drug, in some embodiments.

The end of the jacketed suture furthest from the reservoir can be placed at a location 1114 in a body at which delivery of ions is desired.

A voltage source 1108 can create a voltage across two electrodes 1110 and 1112. Any suitable voltage source can be used as voltage source 1108, and the voltage source can have any suitable voltage (e.g., such as 10 VDC), in some embodiments. Electrodes 1110 and 1112 can be any suitable electrodes. Electrodes 1110 can be placed in reservoir 1106, and electrode 1112 can be placed at or adjacent to location 1114. The polarity applied to the electrodes can depend on the ions being transferred from reservoir 1106 to location 1114. When the ions are positive as shown in FIG. 11, electrode 1110 can be positive and electrode 1112 can be negative. When the ions are negative, electrode 1110 can be negative and electrode 1112 can be positive.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

What is claimed is:

1. A suture, comprising:
a surgical suture material; and
a conducting coating that has been applied to the surgical suture material,
wherein an electrical conductivity of the suture is greater than 500 S/cm per 1964 μm² of cross-sectional area of the suture.

2. The suture of claim 1, wherein an electro-chemical impedance of the suture is less than 10 kOhms at 1 kHz per 1964 μm² of cross-sectional area of the suture.

3. The suture of claim 2, wherein a diameter of the suture is 50-100 μm.

4. The suture of claim 1, wherein the conducting coating includes PEDOT:PSS.

5. The suture of claim 4, wherein the conducting coating further includes metal particles or metal flakes.

6. The suture of claim 1, further comprising: an insulating layer.

7. The suture of claim 6, wherein the insulating layer comprises at least one of Parylene-C, polyurethane, and a silicone elastomer.

8. The suture of claim 6, wherein the insulating layer was applied to the surgical suture material after the conducting coating was applied to the surgical suture material.

9. The suture of claim 1, wherein the suture is capable of being used for wound suturing.

10. The suture of claim 1, wherein the suture is capable of being used for electrophysiological signal acquisition, electrophysiological signal transmission, or electrical stimulation.

11. The suture of claim 1, wherein the suture is capable of being used for power delivery.

12. The suture of claim 1, wherein the suture is capable of being used for ion delivery.

13. A method for forming a suture, comprising:
coating a surgical suture material with a conducting coating to form a conducting suture,
wherein an electrical conductivity of the conducting suture is greater than 500 S/cm per 1964 μm² of cross-sectional area of the conducting suture.

14. The method of claim 13, wherein an electro-chemical impedance of the conducting suture is less than 10 kOhms at 1 kHz per 1964 μm² of cross-sectional area of the conducting suture.

15. The method of claim 14, wherein a diameter of the conducting suture is 50-100 μm.

16. The method of claim 13, wherein the conducting coating includes PEDOT:PSS.

17. The method of claim 13, wherein the conducting coating further includes metal particles or metal flakes.

18. The method of claim 13, further comprising forming an insulating layer on the conducting suture.

19. The method of claim 18, wherein the insulating layer comprises at least one of Parylene-C, polyurethane, and a silicone elastomer.

20. The method of claim 18, wherein the insulating layer was applied to the surgical suture material after the conducting coating was applied to the surgical suture material.

21. The method of claim 13, further comprising using the conducting suture for wound suturing.

22. The method of claim 13, further comprising using the conducting suture for electrophysiological signal acquisition, electrophysiological signal transmission, or electrical stimulation.

23. The method of claim 13, further comprising using the conducting suture for power delivery.

24. The method of claim 13, further comprising using the conducting suture for ion delivery.
